(19) United States
(12) Patent Application Publication
(10) Pub. No.: US 2009/0124626 A1
(43) Pub. Date: May 14, 2009

(54) Pharmaceutical agent comprising insulin resistance improving agent

(75) Inventors: Shoichi Kanda, Tokyo (JP); Ryutaro Nakashima, Kanagawa (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: DAIICHI SANKYO COMPANY, LIMITED, Tokyo (JP)

(21) Appl. No.: 12/079,545

(22) Filed: Mar. 27, 2008

Related U.S. Application Data
(63) Continuation-in-part of application No. PCT/JP2006/319239, filed on Sep. 28, 2006.

(30) Foreign Application Priority Data
Sep. 29, 2005 (JP) 2005-283466

(51) Int. Cl. A61K 31/498; A61K 31/427; A61P 3/10

(52) U.S. Cl. 514/249; 514/369

(57) ABSTRACT

To provide a method for treating diabetes which has an excellent hypoglycemic action and causes minimal adverse drug reactions, there is provided a medicine comprising a DPP-IV inhibitor and an insulin sensitizer in combination.

This is a Continuation-in-Part Application of International Application PCT/JP2006/319239 filed Sep. 28, 2006, incorporated herein by reference in its entirety.

### TECHNICAL FIELD

The present invention relates to a medicine (preferably, a therapeutic and/or prophylactic agent for diabetes) comprising a dipeptidyl peptidase IV inhibitor (DPP-IV inhibitor) and an insulin sensitizer in combination.

Furthermore, the present invention relates to use of the above-mentioned compounds for manufacture of the above-mentioned medicine and a method for prophylactic or therapeutic treatment of the above-mentioned disease comprising administration of the above-mentioned medicine to a homeotherm (preferably, a human).

### BACKGROUND ART

Insulin sensitizers are administered to patients as therapeutic agents for diabetes to decrease blood glucose levels by improving an impaired insulin action. Furthermore, reports have shown that these agents have prophylactic and therapeutic effects against not only diabetes, but also diseases attributable to insulin resistance such as hyperglycemia, impaired glucose tolerance, hypertension, hyperlipemia, diabetic complications, gestational diabetes, and polycystic ovary syndrome and cardiovascular diseases such as atherosclerosis. Examples of currently marketed insulin sensitizers include pioglitazone, rosiglitazone, and so forth. It is thought that these agents exert an effect of improving and impaired insulin action by activating peroxisome proliferator-activated receptor (PPAR) γ, and 5-{4-[(6-methoxy-1-methyl-1H-benzimidazol-2-yl)methoxy]benzyl}-1,3-thiazolidine-2,4-dione and pharmacologically acceptable salts thereof are also known to be insulin sensitizers having a PPAR γ activating action (refer to Patent Documents 1 and 2).

Meanwhile, DPP-IV inhibitors are expected as future therapeutic agents for diabetes because of their action of decreasing blood glucose levels and are disclosed in Patent Documents 3 to 6, for example.

Use of an insulin sensitizer and a DPP-IV inhibitor in combination is described in Patent Document 7, for example. However, effects of the combination use of the specific agents of the present invention are unknown.

[Patent Document 1]

WO00/71540

(U.S. Pat. No. 6,706,746)

[Patent Document 2]

Japanese Patent Application No. 2005-14930

(WO 06/78037; U.S. application Ser. No. 11/795,270)

[Patent Document 3]

WO05/3135 (U.S. Pat. No. 7,326,708)

[Patent Document 4]

WO03/4498 (U.S. Pat. No. 7,125,873 and U.S. Pat. No. 6,698,871)

[Patent Document 5]

WO00/34241

(U.S. Pat. No. 6,166,063)

[Patent Document 6]

WO01/68603

(U.S. Pat. No. 6,395,767)

[Patent Document 7]

WO01/97808

(U.S. Pat. No. 7,078,397; U.S. Pat. No. 7,241,756; US 2007/238756)

### DISCLOSURE OF THE INVENTION

In recognition of the above-mentioned circumstances, the inventors of the present invention assiduously studied about prophylactic and/or therapeutic agents for diabetes that cause minimal adverse drug reactions even in long-term drug treatment and are effective in many diabetic patients. As a result, they found that the foregoing object is achieved by using a DPP-IV inhibitor and an insulin sensitizer in combination, and thus accomplished the present invention.

The present invention provides the followings:

(1) A pharmaceutical composition comprising an insulin sensitizer and a DPP-IV inhibitor in combination;
(2) A pharmaceutical composition, characterized in that an insulin sensitizer is administered, and then a DPP-IV inhibitor is administered;
(3) A pharmaceutical composition, characterized in that adverse drug reactions of a DPP-IV inhibitor are reduced by using an insulin sensitizer and the DPP-IV inhibitor in combination;
(4) The pharmaceutical composition according to any one of the above (1) to (3), which is used for prophylactic and therapeutic treatment of diabetes and has an enhanced hypoglycemic action as compared with administration of either agent alone;
(5) The pharmaceutical composition according to any one of the above (1) to (4), wherein the DPP-IV inhibitor is a pyrazine or adamantyl DPP-IV inhibitor;
(6) The pharmaceutical composition according to any one of the above (1) to (4), wherein the DPP-IV inhibitor is a pyrazine DPP-IV inhibitor;
(7) The pharmaceutical composition according to any one of the above (1) to (4), wherein the DPP-IV inhibitor is MK-0431;
(8) The pharmaceutical composition according to any one of the above (1) to (7), wherein the insulin sensitizer is a thiazolidinedione insulin sensitizer;
(9) The pharmaceutical composition according to any one of the above (1) to (7), wherein the insulin sensitizer is a PPAR γ activator;
(10) The pharmaceutical composition according to any one of the above (1) to (7), wherein the insulin sensitizer is 5-{4-[(6-methoxy-1-methyl-1H-benzimidazol-2-yl)methoxy]benzyl}-1,3-thiazolidine-2,4-dione or a pharmacologically acceptable salt thereof;
(11) The pharmaceutical composition according to any one of the above (1) to (4), wherein the DPP-IV inhibitor is MK-0431, and the insulin sensitizer is 5-{4-[(6-methoxy-1-methyl-1H-benzimidazol-2-yl)methoxy]benzyl}-1,3-thiazolidine-2,4-dione or a pharmacologically acceptable salt thereof;
(12) The pharmaceutical composition according to any one of the above (1) to (11), which is used for prophylactic or therapeutic treatment of a disease attributable to insulin resistance;
(13) The pharmaceutical composition according to any one of the above (1) to (11), which is used for prophylactic or therapeutic treatment of diabetes;
(14) The pharmaceutical composition according to any one of the above (1) to (11), which is used for improvement of glucose tolerance;
(15) The pharmaceutical composition according to any one of the above (1) to (14), which is used to decrease blood glucose levels;
(16) The pharmaceutical composition according to any one of the above (1) to (11), which is used for prophylactic or therapeutic treatment of a cardiovascular disease;
(17) The pharmaceutical composition according to any one of the above (1) to (11), wherein the cardiovascular disease is atherosclerosis;
(18) The pharmaceutical composition according to any one of the above (1) to (17), which is a preparation for administering the respective agents with an interval;
(19) The pharmaceutical composition according to any one of the above (1) to (17), which is a preparation for administering the respective agents simultaneously;
(20) The pharmaceutical composition according to any one of the above (1) to (17), which is a preparation as a fixed combination drug;
(21) The pharmaceutical composition according to any one of the above (1) to (20), which is a preparation for oral administration;
(22) Use of an insulin sensitizer and a DPP-IV inhibitor for manufacture of a medicine comprising an insulin sensitizer and a DPP-IV inhibitor as active ingredients;
(23) The use according to the above (22), wherein the DPP-IV inhibitor is MK-0431;
(24) The use according to the above (22) or (23), wherein the insulin sensitizer is a thiazolidinedione insulin sensitizer;
(25) The use according to the above (22) or (23), wherein the insulin sensitizer is 5-{4-[(6-methoxy-1-methyl-1H-benzimidazol-2-yl)methoxy]benzyl}-1,3-thiazolidine-2,4-dione or a pharmacologically acceptable salt;
(26) The use according to the above (22), wherein the DPP-IV inhibitor is MK-0431, and the insulin sensitizer is 5-{4-[(6-methoxy-1-methyl-1H-benzimidazol-2-yl)methoxy]benzyl}-1,3-thiazolidine-2,4-dione or a pharmacologically acceptable salt; and
(27) A method for treating diabetes, characterized in that an insulin sensitizer and a DPP-IV inhibitor are administered in combination.

In the present invention, the “insulin sensitizer” is a generic term of agents that improve insulin resistance and enhance insulin susceptibility, and examples thereof include pioglitazone (preferably, pioglitazone hydrochloride), rosiglitazone (preferably, rosiglitazone maleate), MCC-555, BMS-298585, AZ-242, LY-519818, R-483, MBX-102, AMG-131 (preferably, para-toluene sulfonates),

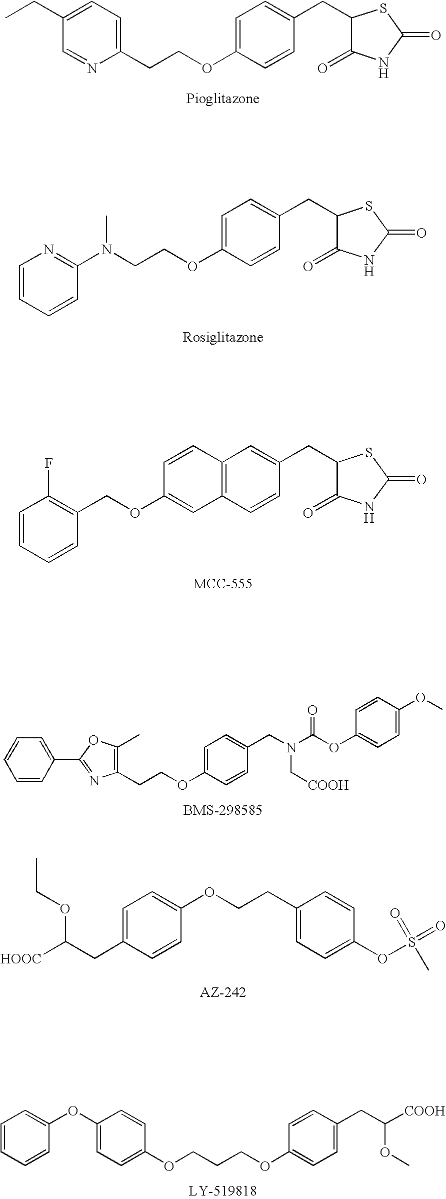

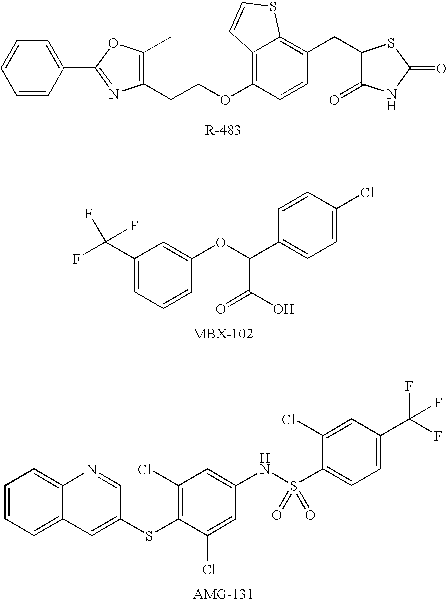

3-(2,4-dichlorobenzyl)-2-methyl-N-(pentylsulfonyl)-3H-benzimidazole-5-carboxamide (FK-614), 5-{4-[(6-methoxy-1-methyl-1H-benzimidazol-2-yl)methoxy]benzyl}-1,3-thiazolidine-2,4-dione (preferably, 5-{4-[(6-methoxy-1-methyl-1H-benzimidazol-2-yl)methoxy]benzyl}-1,3-thiazolidine-2,4-dione hydrochloride), and pharmacologically acceptable salts thereof. Preferred examples thereof include thiazolidinedione insulin sensitizers such as pioglitazone, rosiglitazone, 5-{4-[(6-methoxy-1-methyl-1H-benzimidazol-2-yl)methoxy]benzyl}-1,3-thiazolidine-2,4-dione, and pharmacologically acceptable salts thereof. The thiazolidinedione insulin sensitizers are known to improve insulin resistance by activating peroxisome proliferator-activated receptor (PPAR) γ and are also referred to as PPAR γ activators.

Pioglitazone is a compound described in U.S. Pat. No. 4,687,777. Rosiglitazone is a compound described in U.S. Pat. No. 5,002,953. MCC-555 is a compound described in U.S. Pat. No. 5,594,016. BMS-298585 is a compound described in WO01/21602. AZ-242 is a compound described in WO99/62872. LY-519818 is a compound described in WO02/100813. 3-(2,4-Dichlorobenzyl)-2-methyl-N-(pentylsulfonyl)-3H-benzimidazole-5-carboxamide (FK-614) is a compound described in U.S. Pat. No. 6,166,219. 5-{4-[(6-Methoxy-1-methyl-1H-benzimidazol-2-yl)methoxy]benzyl}-1,3-thiazolidine-2,4-dione and salts thereof are the compound represented by the following structure:

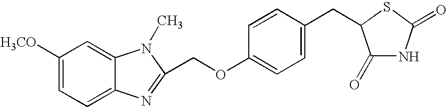

and pharmacologically acceptable salts thereof, and can be produced according to the methods described in Japanese Patent Laid-Open No. 9-295970, EP0745600, U.S. Pat. No. 5,886,014, and WO00/71540.

In the present invention, the “dipeptidyl peptidase IV (DPP-IV) inhibitor” is not particularly limited so long as it is an agent that has actions such as inhibition of DPP-IV and suppression of degradation of GLP-1, and examples thereof include compounds represented by the structural formulas shown below, i.e., (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine and pharmacologically acceptable salts thereof (MK-0431, etc.), which are pyrazine compounds having a pyrazine skeleton described in WO05/3135 and WO03/4498, (2S)-[[(3-hydroxyadamantan-1-yl)amino]acetyl]pyrrolidine-2-carbonitrile and pharmacologically acceptable salts thereof (LAF-237, etc.), which are adamantyl compounds having an adamantyl skeleton described in WO00/34241, (1S,3S,5S)-2-[(2S)-2-amino-2-(3-hydroxyadamantan-1-yl)acetyl]-2-azabicyclo[3.1.0]hexane-3-carbonitrile and pharmacologically acceptable salts thereof (BMS-477118, etc.), which are adamantyl compounds described in WO01/68603, and so forth. (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine and pharmacologically acceptable salts thereof, which are pyrazine compounds, are preferable.

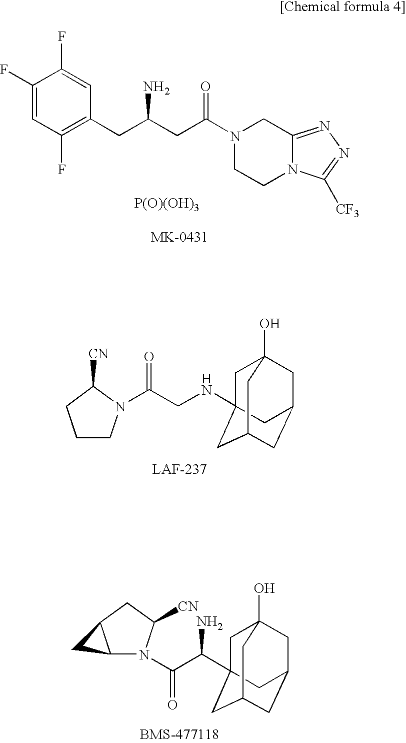

A DPP-IV inhibitor and an insulin sensitizer can be administered in the form of a fixed combination drug. Furthermore, single agents can be simultaneously administered. Furthermore, the single agents can be administered successively with a suitable interval. An interval that is acceptable to achieve an effect obtained by such agents can be confirmed by a clinical or animal experiment.

The pharmaceutical composition of the present invention is administered in various dosage forms. The administration route thereof is not particularly limited and determined depending on the dosage form, patient's age, sex, and other conditions, severity of the disease, and the like. For example, the pharmaceutical composition of the present invention is orally administered in the form of tablet, pill, powder, granule, syrup, solution, suspension, emulsion, granule, or capsule.

These various preparations can be prepared according to conventional methods using known aids usually used in the known field of pharmaceutical preparations such as excipients, binders, disintegrating agents, lubricants, solubilizing agents, flavoring agents, coating agents, in addition to active ingredients.

To prepare in the form of tablets, a broad range of known carriers can be used, and examples thereof include excipients such as lactose, sucrose, sodium chloride, glucose, urea, starch, calcium carbonate, kaolin, crystalline cellulose, and silicic acid, binders such as water, ethanol, propanol, simple syrup, glucose solution, starch solution, gelatin solution, carboxymethylcellulose, shellac, methylcellulose, potassium phosphate, and polyvinylpyrrolidone, disintegrating agents such as dry starch, sodium alginate, powdered agar, laminaran powder, sodium hydrogen carbonate, calcium carbonate, polyoxyethylene sorbitan fatty acid esters, lauryl sodium sulfate, monoglyceride stearate, starch, and lactose, disintegration suppressing agents such as sucrose, stearin, cocoa butter, and hydrogenated oil, absorption promoters such as quaternary ammonium base and lauryl sodium sulfate, moisturizing agents such as glycerine and starch, adsorbents such as starch, lactose, kaolin, bentonite, and colloidal silicic acid, lubricants such as purified talc, stearates, boric acid powder, and polyethylene glycol, and so forth. Furthermore, a tablet can be prepared as a tablet coated with a usual tablet coating, for example, sugar-coated tablet, gelatin-coated tablet, enteric-coated tablet, film-coated tablet, double-layered tablet, or multi-layered tablet, as required.

To prepare in the form of pills, a broad range of carriers known in this field can be used, and examples thereof include excipients such as glucose, lactose, starch, cacao butter, hydrogenated vegetable oil, kaolin, and talc, binders such as powdered gum arabic, powdered tragacanth, gelatin, and ethanol, disintegrating agents such as laminaran agar, and so forth.

Furthermore, if necessary, coloring materials, preservatives, flavors, flavoring agents, sweeteners, and the like, or other pharmaceutical products may be added.

The dose of each therapeutic agent for diabetes used in the present invention and the dosing ratio greatly vary depending on various conditions such as activity of each substance, patient's symptom, age, and body weight (usually assumed to average 60 kg for an adult human).

The insulin sensitizer contained in the above-mentioned pharmaceutical preparation is not particularly limited and suitably selected from a broad range, and an appropriate content is usually 1 to 70% by weight, preferably 1 to 30% by weight of the total composition.

The dose varies depending on the symptom, age, body weight, dosage form, and the like, and the lower limit and the upper limit of the usual daily dose for adults are 0.0001 mg/kg (preferably 0.001 mg/kg, more preferably 0.01 mg/kg); and 30 mg/kg (preferably 3 mg/kg, more preferably 0.3 mg/kg, most preferably 0.03 mg/kg), respectively, which can be administered as one or two doses. Preferable range based on clinical dosages of 0.5, 1.0, 1.5 and 2.0 is 0.5 to 2 mg per day.

The amount of a DPP-IV inhibitor contained in the above-mentioned pharmaceutical preparation is not particularly limited and suitably selected from a broad range, and an appropriate content is usually 1 to 70% by weight, preferably 1 to 30% by weight of the total composition.

For DPP-IV inhibitors, it is contemplated that they be used in their usual effective amounts.

The dose varies depending on the symptom, age, body weight, dosage fomm, and the like, and the lower limit and the upper limit of the usual daily dose for adults are 0.0001 mg/kg (preferably 0.001 mg/kg, more preferably 0.01 mg/kg, most preferably 0.8 mg/kg); and 30 mg/kg (preferably 3 mg/kg, more preferably 1.7 mg/kg), respectively, which can be administered as one to three doses. Total dosages of 50 to 100 mg per day based on available DPP-IV inhibitors, are most preferred.

Regarding the appropriate range for DPP-IV inhibitors in terms of “clinical dosage”, each clinical dose is as follows.

MK-0431: 25 mg, 50 mg, 100 mg/once daily

LAF-237: 50 mg/once or twice daily

BMS-477118: 2.5 mg, 5 mg, 10 mg/once daily

MK-0431 and LAF-237 have been marketed.

In the present invention, the above-mentioned doses of a DPP-IV inhibitor and an insulin sensitizer are administered once daily, or divided into several doses and administered simultaneously or separately at different times.

According to the present invention, an excellent hypoglycemic action is exhibited against high blood glucose levels in diabetes by using a DPP-IV inhibitor and an insulin sensitizer in combination, and diabetes can thereby be prevented or treated effectively. Furthermore, this medicine is also effective for prophylactic and therapeutic treatment of diabetes complications attributable to high blood glucose levels, diseases attributable to insulin resistance such as hyperglycemia, impaired glucose tolerance, hypertension, hyperlipemia, diabetes complications, gestational diabetes, and polycystic ovary syndrome, and cardiovascular diseases such as atherosclerosis. Furthermore, rapid improvement of high blood glucose levels and a stable hypoglycemic action in long-term administration are expected by suitably selecting the type of each agent, administration method, doses, and the like depending on the symptom, and this medicine can be a prophylactic and therapeutic agent for the above-mentioned diseases with very few adverse drug reactions.

### BRIEF DESCRIPTION OF THE DRAWING

FIG. 1 shows a glucose tolerance improving effect by combination use of 5-{4-[(6-methoxy-1-methyl-1H-benzimidazol-2-yl)methoxy]benzyl}-1,3-thiazolidine-2,4-dione hydrochloride (compound A) and MK-0431(compound B) (Test Example 1).

### BEST MODE FOR CARRYING OUT THE INVENTION

The present invention will be explained more specifically with reference to the following examples. However, the scope of the present invention is not limited to these examples.

### Examples

5-{4-[(6-Methoxy-1-methyl-1H-benzimidazol-2-yl)methoxy]benzyl}-1,3-thiazolidine-2,4-dione hydrochloride (compound A), an insulin sensitizer, can be produced according to the methods described in Japanese Patent Laid-Open No. 9-295970, EP0745600, U.S. Pat. No. 5,886,014, and WO00/71540. MK-0431 (compound B), a DPP-IV inhibitor, can be produced according to the methods described in WO05/3135 and WO03/4498.

### Test Example 1

### Glucose Tolerance Improving Effect by Combination Use of 5-{4-[(6-methoxy-1-methyl-1H-benzimidazol-2-yl)methoxy]benzyl}-1,3-thiazolidine-2,4-dione Hydrochloride (Compound A) and MK-0431 (Compound B)

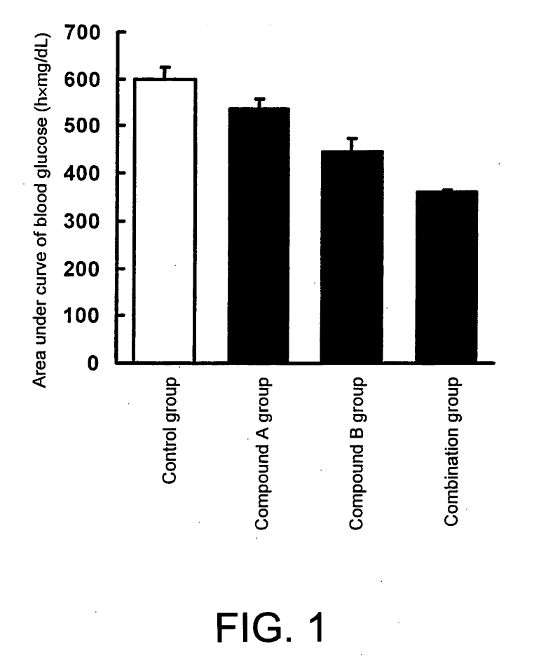

12-week-old male obese Zucker fatty rats (Charles River Laboratories Japan, Inc.) with severe insulin resistance and impaired glucose tolerance were assigned to any of four groups, i.e., the control group, compound A group, compound B group, and combination group (5 animals/group). Food (FR-2, Funabashi Farms Co., Ltd.) was available ad libitum. The compound A group and the combination group were given repeated oral doses of 0.02 mg/kg of compound A, an insulin sensitizer, for one week, the control group and the compound B group were given a vehicle alone for the same period, and then all the groups were fasted overnight. After fasting, a 50% glucose solution (Otsuka Pharmaceutical Factory, Inc.) was orally given at a dose of 2 g/kg to perform a glucose tolerance test. The compound B group and the combination group were given 5 mg/kg of compound B, a DPP-IV inhibitor, one hour before glucose load, and blood samples were collected from the caudal vein of all the individual animals immediately before and 0.5, 1, 1.5, and 2 hours after glucose load to measure blood glucose levels using a fully automated glucose analyzer (Glucoroder-GXT, A&T). The area under curve of blood glucose was calculated for each individual animal with values obtained by substracting the blood glucose level immediately before administration from a blood glucose level measured at each time point. The mean area under curve of blood glucose and the standard error for each group were obtained from these values as shown in FIG. 1. The greater decrease in the area under curve of blood glucose means a higher hypoglycemic action.

As shown in FIG. 1, it can be confirmed that combination use of compound A, an insulin sensitizer, and compound B, a DPP-IV inhibitor, decreases the area under curve of blood glucose without attenuating each other's effect (by two-way analysis of variance). This result suggests that the hypoglycemic action was enhanced by combination use of compound A and compound B.

### Test Example 2

### Glucose Tolerance Improving Effect by Combination Use of 5-{4-[(6-methoxy-1-methyl-1H-benzimidazol-2-yl)methoxy]benzyl}-1,3-thiazolidine-2,4-dione Hydrochloride (Compound A) and MC-0431 (Compound B)

12-week-old male obese Zucker fatty rats (Charles River Laboratories Japan, Inc.) with severe insulin resistance and impaired glucose tolerance are given repeated oral doses of 0.02 mg/kg of compound A, an insulin sensitizer, for one week (feed: FR-2, Funabashi Farms Co., Ltd.), fasted overnight, and orally given a 50% glucose solution (Otsuka Pharmaceutical Factory, Inc.) at a dose of 2 g/kg to perform a glucose tolerance test. The effect of combination use of compound A and compound B is examined by giving 2 mg/kg of compound B, a DPP-IV inhibitor, one hour before glucose load. Drug efficacy is evaluated by blood glucose levels up to two hours after glucose load. The group organization is as follows.

TABLE 1

| | | Administration | |
|---|---|---|---|
| | 1-week repeated | before glucose | No. of |
| | administration | load | animals |
| (i) Control group | Vehicle | Vehicle | 5 |
| (ii) Compound A group | Compound A | Vehicle | 5 |
| (iii) Compound B group | Vehicle | Compound B | 5 |
| (iv) Combination group | Compound A | Compound B | 5 |

It can be confirmed that, the area under curve of blood glucose without attenuating each other's effect (by two-way analysis of variance) decreases in the combination group given compound A, an insulin sensitizer, and compound B, a DPP-IV inhibitor.

The area under curve of blood glucose after administration of glucose is an indicator of glucose tolerance, and an increase in the area under curve indicates impairment of glucose tolerance. Impairment of glucose tolerance is one of the diagnostic criteria of diabetes, and improvement of this condition leads to treatment of diabetes. Therefore, the medicine of the present invention is useful for prophylactic and therapeutic treatment of diabetes because it improves glucose tolerance more effectively than treatment with one agent alone. Furthermore, since adequate effect can be obtained at lower doses of the medicine of the present invention as compared with treatment with each agent alone, adverse drug reactions that appear to be caused by a DPP-IV inhibitor (e.g., anorexia, nausea, liver dysfunction, immunodeficiency, etc.) can be reduced in treatment of diabetes or the like.

### Preparation Examples

(1) Capsule

| | | |
|---|---|---|
| Compound A | 1 | mg |
| Compound B | 25 | mg |
| Lactose | 75 | mg |
| Corn starch | 57 | mg |
| Magnesium stearate | 2 | mg |
| Total | 160 | mg |

All the powder components shown above are well mixed, and sifted through a sieve. 160 mg of the obtained powder is weighed and filled in a gelatin capsule to prepare a capsule.

(2) Tablet

| | | |
|---|---|---|
| Compound A | 1 | mg |
| Compound B | 25 | mg |
| Lactose | 35 | mg |
| Corn starch | 33 | mg |
| Microcrystalline cellulose | 20 | mg |
| Magnesium stearate | 1 | mg |
| Total | 115 | mg |

All the powder components shown above are well mixed, and compressed and molded to tablets each having a weight of 115 mg. If necessary, these tablets may be coated with sugar or a film.

1. A pharmaceutical composition comprising in combination, effective amounts of an insulin sensitizer and of a DPP-IV inhibitor, in a pharmaceutically acceptable carrier, said insulin sensitizer being 5-{4-[(6-methoxy-1-methyl-1H-benzimidazol-2-yl)methoxy]benzyl}-1,3-thiazolidine-2,4-dione or a pharmacologically acceptable salt thereof.

2. The pharmaceutical composition according to claim 1, wherein the DPP-IV inhibitor is an adamantyl DPP-IV inhibitor.

3. The pharmaceutical composition according to claim 1, wherein the DPP-IV inhibitor is a pyrazine DPP-IV inhibitor.

4. The pharmaceutical composition according to claim 1, wherein the DPP-IV inhibitor is MK-0431.

5. The pharmaceutical composition of claim 1 wherein the insulin sensitizer comprises 1-30% percent of the composition and the DPP-IV inhibitor comprises 1-30% of the composition.

6. A method for prophylactic or therapeutic treatment of a disease attributable to insulin resistance comprising administering an effective amount of the composition of claim 1 or 4 to a warm blooded animal in need thereof, and wherein said effective amount of the insulin sensitizer is 0.5 to 2 mg per day.

7. A method for prophylactic or therapeutic treatment of diabetes comprising administering an effective amount of the composition of claim 1 or 4 to a warm blooded animal in need thereof, and wherein said effective amount of the insulin sensitizer is 0.5 to 2 mg per day.

8. A method for improvement of glucose tolerance comprising administering an effective amount of the composition of claim 1 or 4 to a warm blooded animal in need thereof, and wherein said effective amount of the insulin sensitizer is 0.5 to 2 mg per day.

9. A method for decreasing blood glucose levels comprising administering an effective amount of the composition of claim 1 or 4 to a warm blooded animal in need thereof, and wherein said effective amount of the insulin sensitizer is 0.5 to 2 mg per day.

10. A method for prophylactic or therapeutic treatment of a cardiovascular disease comprising administering an effective amount of the composition of claim 1 or 4 to a warm blooded animal in need thereof, and wherein said effective amount of the insulin sensitizer is 0.5 to 2 mg per day.

11. The method of claim 10 wherein the cardiovascular disease is atherosclerosis.

12. A method for improving blood glucose tolerance, comprising administering 0.5 to 2 mg of an insulin sensitizer and an effective amount of an DPP-IV inhibitor, in combination, to a warm blooded animal in need thereof, and wherein the insulin sensitizer is 5-{4-[(6-methoxy-1-methyl-1H-benzimidazol-2-yl)methoxy]benzyl}-1,3-thiazolidine-2,4-dione or a pharmacologically acceptable salt thereof.

13. A method for improving blood glucose tolerance, comprising administering 0.5 to 2 mg of an insulin sensitizer and an effective amount of DPP-IV inhibitor, separately, to a warm blooded animal in need thereof, and wherein the insulin sensitizer is 5-{4-[(6-methoxy-1-methyl-1H-benzimidazol-2-yl)methoxy]benzyl}-1,3-thiazolidine-2,4-dione or a pharmacologically acceptable salt thereof.

14. The method of claim 12 or 13 wherein the DPP-IV inhibitor is MK-0431 and its effective amount is 25 to 100 mg/daily.

15. The method of claim 12 or 13 wherein the DPP-IV inhibitor is LAF-237 and its effective amount is 50 to 100 mg/daily.

16. The method of claim 12 or 13 wherein the DPP-IV inhibitor is BMS 477118 and its effective amount is 2.5 to 10 mg/daily.
